(19) United States
(12) Patent Application Publication
(10) Pub. No.: US 2014/0037507 A1
(43) Pub. Date: Feb. 6, 2014

(54) ORGANIC COMPOUND PYROLYSIS FLASHOVER ENERGY-SAVING REGENERATION TREATMENT SYSTEM

(71) Applicant: Hsiao-Lun WANG, New Taipei City (TW)

(72) Inventor: Hsiao-Lun WANG, New Taipei City (TW)

(21) Appl. No.: 13/562,390

(22) Filed: Jul. 31, 2012

(51) Int. Cl. B01J 8/00; B01D 35/00; A61L 2/07; G05D 27/00

(52) U.S. Cl. 422/106

(57) ABSTRACT

An organic compound pyrolysis flashover energy-saving regeneration treatment system for remaking organic waste such as family\industrial garbage, sewer mud, agricultural waste and livestock excreta etc. to become liquid and solid regenerated organic compounds by using a waste pre-treatment unit, an organic waste steaming sterilization unit, an organic waste pyrolysis flashover unit, a green energy generation & steam regeneration unit, a solid-liquid organic compound separation unit, a liquid organic compound regeneration unit and a solid organic compound regeneration unit, which compounds are used as organic fertilizer or feed for livestock farming, the system can effectively achieve the objects of recovering resources, purifying environment, saving energy and reducing carbon and regeneration of organic waste for reuse, it further can increase the efficiency of environmental conservation.

### BACKGROUND OF THE INVENTION

1. Field of the Invention

The present invention relates to a technique concerning green energy, environmental conservation and recovering and regeneration for use of organic compound, and especially to “an organic compound pyrolysis flashover energy-saving regeneration treatment system” to remake organic waste to become liquid regenerated organic compound and solid regenerated organic compound to be used as organic fertilizer or feed for livestock farming.

2. Description of the Prior Art

Normal waste mostly is treated by the modes of burying, incinerating and recovering, the treatment of burying is subjected to pollution of underground water, this does not accord with the efficiency of environmental conservation; thereby except that a part of the special waste must be buried in solid state, most of the waste is treated by incinerating and recovering to reduce the amount to be buried in a garbage field. Among various kinds of wastes, family garbage food, industrial garbage, sewer mud, agricultural waste and livestock excreta etc. contain plenty of organic ingredients, they are the main factor that make bad odor of garbage and generate disease vector; hence, it can be more likely to dissolve the problem of environment pollution by separating the organic waste from other waste to allow the organic waste to be recovered, and meantime to alleviate the burdens of incinerators and burying grounds.

However, the conventional treatment process for recovering and fermenting organic waste to become piled fertilizer needs a very long time, and may breed mosquitoes, create bad odor to make second time pollution of the ambient circumstances. Moreover, the mode of treatment of fermenting the piled fertilizer is not able to eliminate harmful elements such as the disease bacteria and toxic heavy metal elements etc. in the organic waste; this induces the undesired cases of pollution of soil and poison damage against crops, and finally affects human physical health through the process of transmission of food chain.

In view of the above, the inventor of the present invention provides “an organic compound pyrolysis flashover energy-saving regeneration treatment system” based on his professional experience of years and knowledge from studying in related domain, the system can remake the organic waste such as family garbage food, industrial garbage, sewer mud, agricultural waste and livestock excreta etc. to become liquid regenerated organic compound and solid regenerated organic compound to be used as organic fertilizer or feed for livestock farming, and can effectively create green energy source, further can achieve the objects of recovering resources, purifying environment, saving energy and reducing carbon and regeneration for use.

### SUMMARY OF THE INVENTION

The object of the present invention is to provide “an organic compound pyrolysis flashover energy-saving regeneration treatment system” to remake the organic waste such as family garbage food, industrial garbage, sewer mud, agricultural waste and livestock excreta etc. to become liquid regenerated organic compound and solid regenerated organic compound by using a waste pre-treatment unit, an organic waste steaming sterilization unit, an organic waste pyrolysis flashover unit, a green energy generation & steam regeneration unit, a solid-liquid organic compound separation unit; a liquid organic compound regeneration unit and a solid organic compound regeneration unit for making liquid regenerated organic compound and solid regenerated organic compound, to be used as organic fertilizer or feed for livestock farming; the system can effectively create green energy source, and can achieve the objects of recovering resources, purifying environment, saving energy and reducing carbon and regeneration of organic waste for reuse, it further can reduce the gross treatment amount of garbage burying and incinerating, and increase the efficiency of environmental conservation.

In order to achieve the above stated objects, the “organic compound pyrolysis flashover energy-saving regeneration treatment system” of the present invention comprises:

a waste pre-treatment unit for screening and removing the inorganic waste from waste, and for crushing the solid organic waste to produce the solid organic waste raw material, after that, neutralizing the raw material with the liquid organic waste raw material and solidifying them to form organic waste raw material;

an organic waste steaming sterilization unit for neutralizing the neutralized and solidified organic waste raw material with additive and heating and steam cooking them to a specific temperature to kill disease bacterial;

an organic waste pyrolysis flashover unit for pouring a combustion catalyst of a specific temperature into the organic waste raw material with the specific temperature mentioned in the former paragraph, in order to arouse pyrolyzing flashing over of organic biologic material such as fat, protein etc. and meantime to burn and remove toxic heavy metal elements;

a green energy generation & steam regeneration unit to transduce the heat energy created by pyrolyzing flashing over of the above stated organic biologic material into green energy, and to recover condensed water to form regeneration flasher steam to supply various heat consuming units for circulating using;

an organic material solid-liquid separation unit for filtering and separating the abovementioned organic compound with its toxic elements removed through burning by the organic waste pyrolysis flashover unit to become liquid organic water and solid organic compound;

a liquid organic compound regeneration unit to condense and extract the liquid organic water to form liquid organic compound which is neutralized with additive to become regenerated liquid organic compound product;

a solid organic compound regeneration unit for treating, regenerating the solid organic compound separated from the liquid organic compound under a constant temperature through neutralization with additive and thereafter atomizing, dehumidifying and granulating the solid organic compound to become regenerated solid organic compound product; and

a remote monitoring and automatic adjustment unit which can collect and transmit values and signals including pressure, flow, level of liquid, temperature and humidity etc. to a central control system for automatic adjusting and control.

The above stated units are provided among a plurality of tanks of them with mutually communicated through airtight pipes, each airtight pipe is provided thereon with a pusher set; each pusher set has its unique function, and can push the compound produced to its next unit. The organic waste is made regenerated liquid organic compound and regenerated solid organic compound through the waste pre-treatment unit, the organic waste steaming sterilization unit, the organic waste pyrolysis flashover unit, the green energy generating & steam regeneration unit, the solid-liquid organic compound separation unit, the liquid organic compound regeneration unit and the solid organic compound regeneration unit for making liquid regenerated organic compound and solid regenerated organic compound, the compounds made are used as organic fertilizer or feed for livestock farming, and can achieve the objects of recovering resources, purifying environment, saving energy and reducing carbon and regeneration for use.

The followings are descriptions of the practice mode of the units:

In practicing the “waste pre-treatment unit”, the unit at least includes a foreign particles inspection set, an auto crusher set, a solid raw material tank, a liquid raw material tank, a solid-liquid mixed tank and a high pressure heatproof pusher set, in which, the foreign particles inspection set can do screening, separating and removing the inorganic waste from waste material, the solid organic waste of different types and sizes after being screened such as family garbage food, industrial garbage, agricultural waste etc. are delivered into the auto crusher set to be crushed, to form the solid organic waste raw material meeting the size of the granules treated subsequently, the granules then are delivered to the solid raw material tank for concentrating and storing, and then a solid feeding pump automatically quantitatively pushes the solid organic waste raw material into the solid-liquid mixed tank according to the requirement of the manufacturing process; and the liquid organic waste raw material such as the sewer mud and the livestock excreta etc. are concentrated and stored in the liquid raw material tank, and then a liquid feeding pump automatically quantitatively pushes the liquid organic waste raw material into the solid-liquid mixed tank according to the requirement of the manufacturing process to be neutralized and stirred together with the solid organic waste; then the regeneration flasher steam produced by using the green energy generation & steam regeneration unit and/or the solid organic waste raw material formed after synchronically heating and solidifying by an auxiliary heater are/is used to have the solid organic waste raw material automatically quantitatively pushed to the organic waste steaming sterilization unit through the high pressure heatproof pusher set.

In practicing, “the organic waste steaming sterilization unit” at least includes a single or a plurality of high temperature steaming sterilization set(s), a single or a plurality of coaxial injection vortex propeller(s), an additive supply set and a high pressure heatproof pusher set, wherein the additive supply set can take advantage of the coaxial injection vortex propeller(s) to synchronically and uniformly spray the additive and the regeneration flasher steam into the high temperature steaming sterilization set(s) to have them neutralized with the solid organic waste raw material, and to have them heated to a specific high temperature synchronically by using the regeneration flasher steam and/or the auxiliary heater to kill the disease bacterial; and the high pressure heatproof pusher set automatically quantitatively pushes (under a constant temperature) the organic waste raw material having a specific temperature to the organic waste pyrolysis flashover unit via a heating airtight pipe.

And in practicing, “the organic waste pyrolysis flashover unit” at least includes a single or a plurality of high pressure pyrolysis combustion set(s), a single or a plurality of coaxial injection vortex propeller(s), a combustion catalyst supply set, a heat exchanger set and a high pressure heatproof pusher set, wherein the combustion catalyst supply set can heat combustion catalyst through the heat exchanger set by using the regeneration flasher steam and/or the auxiliary heater to form a combustion catalyst with a specific temperature, and uniformly sprays and injects the combustion catalyst with the specific temperature into the high pressure pyrolysis combustion set(s) through the coaxial injection vortex propeller(s) to be neutralized with the organic waste raw material with a specific temperature, to thereby arouse transient pyrolyzing flashing over of the organic biologic material such as fat, protein etc. in the organic waste raw material, and after synchronically having the toxic heavy metal elements removed by burning, the organic compound having been dealt with pyrolyzing, flashing over and detoxification is conveyed by the high pressure heatproof pusher set to the organic compound solid-liquid separation unit automatically and quantitatively via an airtight pipe to be dealt with the operation in the next step; while the high temperature and pressure steam created during pyrolyzing flashing over is conveyed to other units for heating through a steam pipe, or is conveyed to the green energy generation & steam regeneration unit used as kinetic energy for green energy generation.

In practicing, “the green energy generation & steam regeneration unit” at least includes a cogeneration set and a steam regeneration set, the cogeneration set can transduce the high temperature and pressure steam created synchronically by the above mentioned organic waste pyrolysis flashover unit to become a green energy supplying system for self use or selling for people; and more, the high temperature condensation water created after green energy generation is recovered by using a steaming regeneration set, and is regenerated to become flasher steam to be supplied to various units for circulating using.

And in practicing, “the organic compound solid-liquid separation unit” at least includes a high temperature solid-liquid separation set, a high pressure airtight grinder set and a high pressure heatproof pusher set, wherein the high temperature solid-liquid separation set can filter and separate the liquid organic water and the solid organic compound in the abovementioned organic compound having been treated with the pyrolyzing flashing over and toxic elements removing operation; the liquid organic water after separation is sent to the liquid organic compound regeneration unit via an airtight pipe, while the solid organic compound after separation is sent to the high pressure airtight grinder set via an airtight pipe to be ground to get fine granules of which the granule size meets the requirement of the subsequent operation, then is pushed automatically and quantitatively to the solid organic compound regeneration unit through the high pressure heatproof pusher set via an airtight pipe.

And in practicing, “the liquid organic compound regeneration unit” at least includes a liquid extraction set, a quantitative pumping set, a additive supply set and neutralization and regulating tank, wherein the liquid extraction set condenses and extracts the liquid organic water to form liquid organic compound for concentrating and storing, and then the compound is pushed through the quantitative pumping set to the neutralization and regulating tank via an airtight pipe; and the additive supply set automatically quantitatively pushes the additive together with a liquid organic neutralization and regulating agent into the neutralization and regulating tank via an airtight pipe to form regenerated liquid organic compound product.

Further in practicing, “the solid organic compound regeneration unit” at least includes a single or a plurality of organic compound regeneration set(s), a single or a plurality of coaxial inject on vortex propeller(s), an additive supply set and an atomizing and dehumidifying granulation set, the additive supply set can take advantage of the coaxial injection vortex propeller(s) to synchronically and uniformly spray the additive and the regeneration flasher steam into the organic compound regeneration set to be neutralized together with the solid organic compound, and after synchronically having them accepted regeneration treatment by using the regeneration flasher steam and/or the auxiliary heater, the high pressure and high temperature solid organic is fast sprayed into a low temperature and low pressure atomizing and dehumidifying granulation set, to synchronically atomize dehumidify and granulate the solid organic compound according to the principle of transient cooling and dehumidifying, thus solid regenerated organic compound product can be made.

And in practicing, “the remote monitoring and automatic adjustment unit” at least includes a plurality of sensors provided respectively at the above mentioned units, these sensors collect and transmit value signals of pressures, flows, liquid levels, temperatures and humidity etc. respectively sensed by the sensors to a central control system for automatic adjustment, and for operation of remote monitoring for the professional personnel in a safe protected space. The above stated a plurality of sensors include but are not limited to: pressure sensors, flow sensors, liquid level sensors, temperature sensors and humidity sensors.

As compared to the prior art, the present invention can remake organic waste such as family garbage food, industrial garbage, sewer mud, agricultural waste and livestock excreta etc. to become liquid regenerated organic compound and solid regenerated organic compound through a waste pre-treatment unit, an organic waste steaming sterilization unit, an organic waste pyrolysis flashover unit, a green energy generation & steam regeneration unit, a solid-liquid organic compound separation unit, a liquid organic compound regeneration unit and a solid organic compound regeneration unit, to be used as organic fertilizer or feed for livestock farming, the present invention can effectively create green energy source, and can achieve the objects of recovering resources, purifying environment, saving energy and reducing carbon and regeneration for use, it further can reduce the gross treatment amount of garbage burying and incinerating, and increase the efficiency of environmental conservation.

The present invention will be apparent in its content, structural combination, effect to be achieved and entire mode of operation after reading the detailed description of the preferred embodiment thereof in reference to the accompanying drawings.

### BRIEF DESCRIPTION OF THE DRAWINGS

FIG. 1 is a schematic block diagram showing the organic compound pyrolysis flashover energy-saving regeneration treatment system of the present invention;

FIG. 2 is a plane view showing an embodiment in detail of the organic compound pyrolysis flashover energy-saving regeneration treatment system of the present invention.

### DETAILED DESCRIPTION OF THE PREFERRED EMBODIMENT

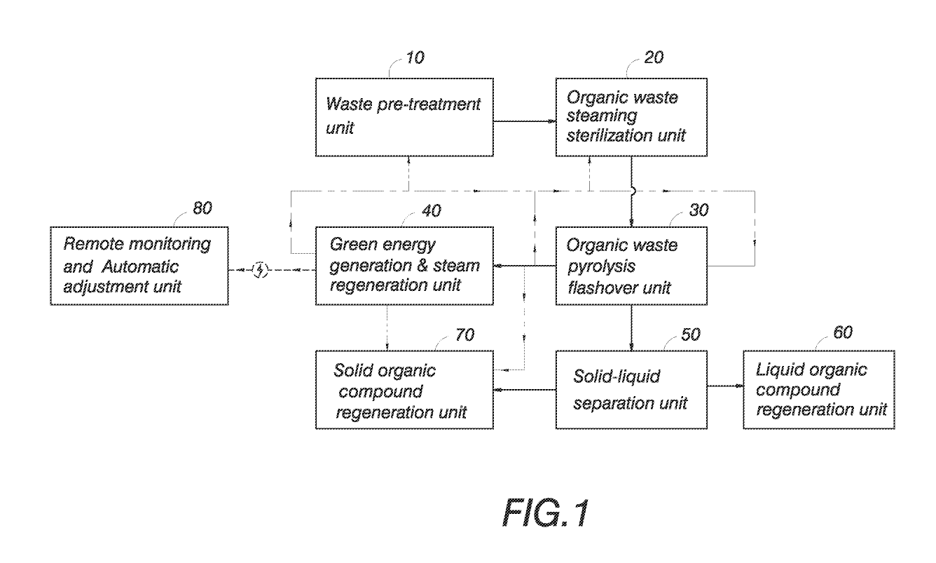

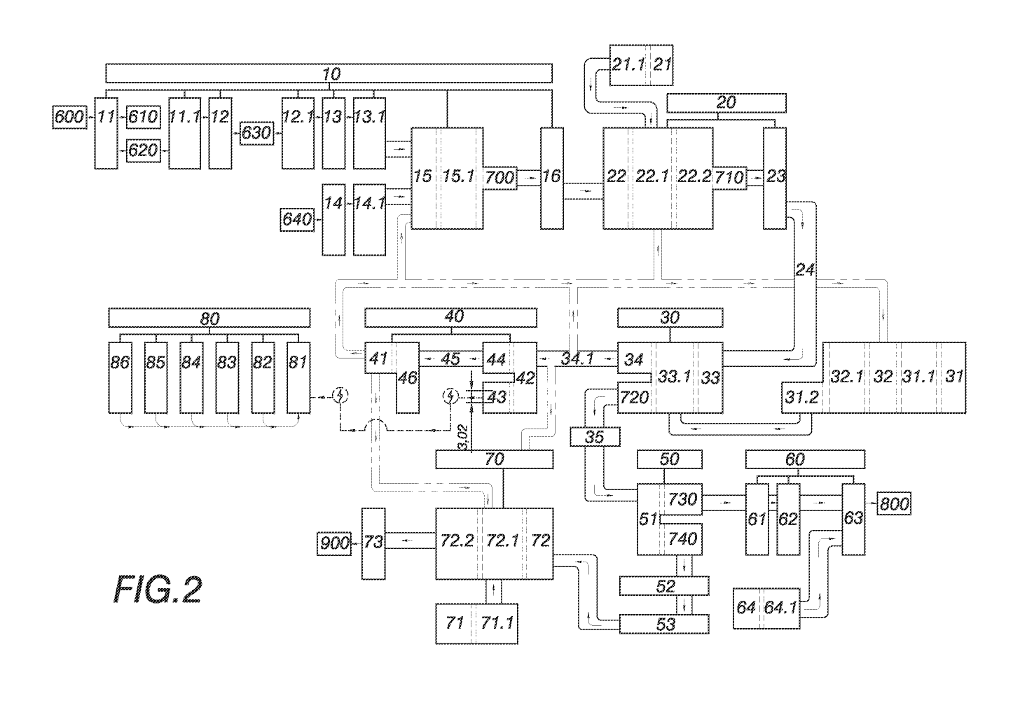

The followings describe the preferred embodiment being put in practice according to the technical measures of the present invention and describe by way of example only with reference to the accompanying drawings FIGS. 1 to 2, wherein:

Referring firstly to FIG. 1, “the organic compound pyrolysis flashover energy-saving regeneration treatment system” of the present invention includes a waste pre-treatment unit 10, an organic waste steaming sterilization unit 20, an organic waste pyrolysis flashover unit 30, a green energy generation & steam regeneration unit 40, a solid-liquid organic compound separation unit 50, a liquid organic compound regeneration unit 60, a solid organic compound regeneration unit 70 and a remote monitoring and automatic adjustment unit 80; a plurality of tanks of the above mentioned units are provided among them airtight conveying pipes for mutually communicating, each conveying pipe is provided thereon with a pusher set having its unique function, and can push the compound produced to its next unit.

The organic waste can effectively create green energy source, and can be made regenerated liquid organic compound and regenerated solid organic compound through the waste pre-treatment unit 10, the organic waste steaming sterilization unit 20, the organic waste pyrolysis flashover unit 30, the green energy generating & steam regeneration unit 40, the solid-liquid organic compound separation unit 50, the liquid organic compound regeneration unit 60 and the solid organic compound regeneration unit 70 and through remote control operation of the remote monitoring and automatic adjustment unit 80, the compounds made are used as organic fertilizer or feed for livestock farming, and can achieve the objects of recovering resources, purifying environment, saving energy and reducing carbon and regeneration for use. The followings describe the way of practicing the above units with reference to drawings:

As shown in FIG. 2, when in practicing “the waste pre-treatment unit 10”, a foreign particles inspection set 11 can do screening, separating and removing inorganic waste 610 from waste material 600; after screening, solid organic waste 620 of different types and sizes such as family garbage food, industrial garbage, agricultural waste etc. are delivered with a conveyer 11.1 into an auto crusher set 12 to be crushed to form solid organic waste raw material 630 with granule size meeting the size of the granules treated subsequently, the solid organic waste raw material 630 then is delivered to a solid raw material tank 13 for concentrating and storing by using a feeder 12.1, and then a solid feeding pump 13.1 automatically quantitatively pushes the solid organic waste raw material 630 into a solid-liquid mixed tank 15 according to the requirement of the manufacturing process through an airtight pipe; and liquid organic waste raw material 640 such as the sewer mud and the livestock excreta etc. are concentrated and stored in a liquid raw material tank 14, and then a liquid feeding pump 14.1 automatically quantitatively pushes the liquid organic waste raw material 640 through an airtight pipe into the solid-liquid mixed tank 15 according to the requirement of the manufacturing process to be neutralized and stirred together with the solid organic waste raw material 630; then regeneration flasher steam 41 produced by using the green energy generation & steam regeneration unit 40 and/or solid organic waste raw material 700 formed after synchronically heating and solidifying by an auxiliary heater 15.1 are/is used to have the solid organic waste raw material 700 automatically quantitatively pushed to the organic waste steaming sterilization unit 20 through a high pressure heatproof pusher set 16 via an airtight pipe.

In practicing of “the organic waste steaming sterilization unit 20”, an additive supply set 21 can take advantage of a coaxial injection vortex propeller 22.1 to synchronically and uniformly spray the additive 21.1 and the regeneration flasher steam 41 into a high temperature steaming sterilization set 22 to have them neutralized with the solid organic waste raw material 700, and to have them heated to a specific high temperature synchronically by using the regeneration flasher steam 41 and/or an auxiliary heater 22.2 to kill the disease bacterial; and a high pressure heatproof pusher set 23 automatically quantitatively pushes (under a constant temperature) organic waste raw material 710 having a specific temperature to the organic waste pyrolysis flashover unit 30 via a heating airtight pipe 24, in order to proceed to the next treatment operation.

And in practicing of “the organic waste pyrolysis flashover unit 30”, a combustion catalyst supply set 31 can heat combustion catalyst 31.1 through a heat exchanger set 32 by using the regeneration flasher steam 41 and/or the auxiliary heater 32.1 to form a combustion catalyst 31.2 with a specific temperature, and uniformly sprays and injects the combustion catalyst with the specific temperature into a high pressure pyrolysis combustion set 33 through a coaxial injection vortex propeller 33.1 to be neutralized with the organic waste raw material 710, to thereby arouse transient pyrolyzing flashing over of the organic biologic material such as fat, protein . . . etc. contained in the organic waste raw material 710, and after synchronically having the toxic heavy metal elements removed by burning, organic compound 720 having been dealt with pyrolyzing, flashing over and detoxification is conveyed by a high pressure heatproof pusher set 35 to the organic compound solid-liquid separation unit 50 automatically and quantitatively via an airtight pipe to be dealt with the operation in the next step; while high temperature and pressure steam 34 created during pyrolysis flashing over is conveyed to other units for heating through a steam pipe 34.1, or is conveyed to the green energy generation & steam regeneration unit 40 used as kinetic energy for green energy generation.

In practicing “the green energy generation & steam regeneration unit 40”, a cogeneration set 42 can transduce the high temperature and pressure steam 34 created synchronically by the above mentioned organic waste pyrolysis flashover unit 30 to become a supplying system of green energy 43 for self use or selling for people; and more, high temperature condensation water 44 created after generation of the green energy 43 is conveyed through a condensation water pipe 45 and is conveyed to and recovered by a steaming regeneration set 46, and is regenerated to become flasher steam 41 after separation of steam and water to be supplied to various units for circulating using.

And in practicing of “the organic compound solid-liquid separation unit 50”, a high temperature solid-liquid separation set 51 can filter and separate liquid organic water 730 and solid organic compound 740 inorganic compound 720, the liquid organic water 730 after separation is sent to the liquid organic compound regeneration unit 60 via an airtight pipe to proceed to the next treatment operation; while solid organic compound 740 after separation is sent to a high pressure airtight grinder set 52 via an airtight pipe to be ground to get fine granules of which the granule size meets the requirement of the subsequent operation, then is pushed automatically and quantitatively to the solid organic compound regeneration unit 70 through a high pressure heatproof pusher set 53 via an airtight pipe to proceed to the next treatment operation.

And in practicing “the liquid organic compound regeneration unit 60”, a liquid extraction set 61 condenses and extracts the liquid organic water 730 to form liquid organic compound for concentrating and storing, and then the compound is pushed through a quantitative pumping set 62 to a neutralization and regulating tank 63 via an airtight pipe; and an additive supply set 64 automatically quantitatively pushes additive 64.1 together with a liquid organic stirring, neutralization and regulating agent into the neutralization and regulating tank 63 via an airtight pipe to form regenerated liquid organic compound product 800.

Further in practicing of “the solid organic compound regeneration unit 70”, an additive supply set 71 can take advantage of a coaxial injection vortex propeller 72.1 to synchronically and uniformly spray additive 71.1 and the regeneration flasher steam 41 into an organic compound regeneration set 72 to be neutralized together with the solid organic compound 740, and after synchronically having them accepted regeneration treatment by using the regeneration flasher steam 41 and/or an auxiliary heater 72.2, the high pressure and high temperature solid organic compound 740 is fast sprayed into a low temperature and low pressure atomizing and dehumidifying granulation set 73 via an airtight pipe, to synchronically atomize, dehumidify and granulate the solid organic compound 740 according to the principle of high temperature transient cooling and dehumidifying, thus solid regenerated organic compound product 900 can be made.

And in practicing of “the remote monitoring and automatic adjustment unit 80”, a plurality of sensors provided respectively at the above mentioned units are used to collect and transmit the value signals of pressures, flows, liquid levels, temperatures and humidity etc. respectively sensed by the sensors to a central control system 81 for automatic adjustment, and for operation of remote monitoring for the professional personnel in a safe protected space. The above stated a plurality of sensors include but are not limited to: a pressure sensor 82, a flow sensor 83, a liquid level sensor 84, a temperature sensor 85 and a humidity sensor 86.

The specification above and the drawings depicted are only for illustrating preferred embodiment of the present invention, and not for giving any limitation to the scope of the present invention. It will be apparent to those skilled in this art that various equivalent modifications without departing from the spirit of this invention in structure, characteristic as well as installation shall fall within the scope of the appended claims and are intended to form part of this invention.

1. A organic compound pyrolysis flashover energy-saving regeneration treatment system comprising:
a waste pre-treatment unit for screening and removing inorganic waste from waste, and for crushing solid organic waste to produce solid organic waste raw material, after that, neutralizing said raw material with liquid organic waste raw material and solidifying them to form organic waste raw material;
an organic waste steaming sterilization unit for neutralizing said organic waste raw material having been neutralized and solidified with additive and heating and steam cooking them to a specific temperature to kill disease bacterial;
an organic waste pyrolysis flashover unit for pouring a combustion catalyst of a specific temperature into said organic waste raw material with specific temperature, in order to arouse pyrolyzing flashing over of organic biologic material such as fat, protein . . . etc. and meantime to burn and remove toxic heavy metal elements;
a green energy generation & steam regeneration unit to transduce heat energy created by pyrolyzing flashing over of said organic biologic material into green energy, and to recover condensed water to form regeneration flasher steam to supply various heat consuming units for circulating using;
an organic material solid-liquid separation unit for filtering and separating said organic compound with its toxic elements removed through burning by said organic waste pyrolysis flashover unit to become liquid organic water and solid organic compound;
a liquid organic compound regeneration unit to condense and extract said liquid organic water to form liquid organic compound which is neutralized with additive to become regenerated liquid organic compound product;
a solid organic compound regeneration unit for treating, regenerating said solid organic compound separated from said liquid organic compound under a constant temperature through neutralization with additive and thereafter atomizing, dehumidifying and granulating said solid organic compound to become regenerated solid organic compound product; and
a remote monitoring and automatic adjustment unit which collects and transmits values and signals including pressure, flow, level of liquid, temperature and humidity etc. to a central control system for automatic adjusting and control.

2. The organic compound pyrolysis flashover energy-saving regeneration treatment system as claimed in claim 1, said waste pre-treatment unit includes a foreign particles inspection set, an auto crusher set, a solid raw material tank, a liquid raw material tank, a solid-liquid mixed tank and a high pressure heatproof pusher set, wherein:
said foreign particles inspection set performs screening, separation and removing of inorganic waste from waste material, said solid organic waste after being screened are delivered into said auto crusher set to be crushed, to form solid organic waste raw material to be delivered to said solid raw material tank for concentrating and storing, and then to be pushed automatically quantitatively into said solid-liquid mixed tank;
and liquid organic waste raw material is concentrated and stored in said liquid raw material tank, and then is pushed automatically quantitatively into said solid-liquid mixed tank to be neutralized and stirred together with said solid organic waste; then regeneration flasher steam produced by using said green energy generation & steam regeneration unit and/or organic waste raw material formed after synchronically heating and solidifying by an auxiliary heater are/is used to have said solid organic waste raw material automatically quantitatively pushed to said organic waste steaming sterilization unit through said high pressure heatproof pusher set.

3. The organic compound pyrolysis flashover energy-saving regeneration treatment system as claimed in claim 1, said organic waste steaming sterilization unit includes a single or a plurality of high temperature steaming sterilization set(s), a single or a plurality of coaxial injection vortex propeller(s), an additive supply set and a high pressure heatproof pusher set, wherein:
said additive supply set take advantage of said coaxial injection vortex propeller(s) to synchronically and uniformly spray said additive and said regeneration flasher steam into said high temperature steaming sterilization set(s) to have them neutralized with said solid organic waste raw material, and to have them heated to a specific high temperature synchronically by using said regeneration flasher steam and/or said auxiliary heater to kill disease bacterial; and said high pressure heatproof pusher set automatically quantitatively pushes (under a constant temperature) said organic waste raw material having a specific temperature to said organic waste pyrolysis flashover unit.

4. The organic compound pyrolysis flashover energy-saving regeneration treatment system as claimed in claim 1, said organic waste pyrolysis flashover unit includes a single or a plurality of high pressure pyrolysis combustion set(s), a single or a plurality of coaxial injection vortex propeller(s), a combustion catalyst supply set, a heat exchanger set and a high pressure heatproof pusher set, wherein:
said combustion catalyst supply set heats combustion catalyst through said heat exchanger set by using said regeneration flasher steam and/or said auxiliary heater to form a combustion catalyst with a specific temperature, and uniformly sprays and injects said combustion catalyst with said specific temperature into said high pressure pyrolysis combustion set(s) through said coaxial injection vortex propeller(s) to be neutralized with said organic waste raw material having a specific temperature, to thereby arouse transient pyrolyzing flashing over of said organic biologic material in said organic waste raw material, and after synchronically having toxic heavy metal elements removed by burning, said organic compound is conveyed by said high pressure heatproof pusher set to said organic compound solid-liquid separation unit automatically and quantitatively via an airtight pipe; while high temperature and pressure steam created during pyrolysis flashing over is conveyed to others of said units for heating, or is conveyed to said green energy generation & steam regeneration unit.

5. The organic compound pyrolysis flashover energy-saving regeneration treatment system as claimed in claim 1, said green energy generation & steam regeneration unit includes a cogeneration set and a steam regeneration set, wherein:
said cogeneration set transduces high temperature and pressure steam created synchronically by said organic waste pyrolysis flashover unit to become a green energy supplying system for self use or selling for people; and more, high temperature condensation water created after green energy generation is recovered by using said steaming regeneration set, and is regenerated to become flasher steam to be supplied to said units for circulating using.

6. The organic compound pyrolysis flashover energy-saving regeneration treatment system as claimed in claim 1, said organic compound solid-liquid separation unit includes a high temperature solid-liquid separation set, a high pressure airtight grinder set and a high pressure heatproof pusher set, wherein:
wherein said high temperature solid-liquid separation set filters and separates said liquid organic water and said solid organic compound in said organic compound having been treated with said pyrolyzing flashing over and toxic elements removing operation; said liquid organic water after separation is sent to said liquid organic compound regeneration unit, while said solid organic compound is sent to said high pressure airtight grinder set to be ground to get fine granules, then is pushed automatically and quantitatively to said solid organic compound regeneration unit through said high pressure heatproof pusher set.

7. The organic compound pyrolysis flashover energy-saving regeneration treatment system as claimed in claim 1, said liquid organic compound regeneration unit includes a liquid extraction set, a quantitative pumping set, a additive supply set and neutralization and regulating tank, wherein:
said liquid extraction set condenses and extracts said liquid organic water to form liquid organic compound for concentrating and storing, and then said compound is pushed through said quantitative pumping set via an airtight pipe to said neutralization and regulating tank; and said additive supply set automatically quantitatively pushes additive together with a liquid organic neutralization and regulating agent into said neutralization and regulating tank via an airtight pipe to form regenerated liquid organic compound product.

8. The organic compound pyrolysis flashover energy-saving regeneration treatment system as claimed in claim 1, said solid organic compound regeneration unit includes a single or a plurality of organic compound regeneration set(s), a single or a plurality of coaxial injection vortex propeller(s), an additive supply set and an atomizing and dehumidifying granulation set, wherein:
said additive supply set takes advantage of said coaxial injection vortex propeller(s) to synchronically and uniformly spray additive and said regeneration flasher steam into said organic compound regeneration set to be neutralized together with said solid organic compound, and after synchronically having them accepted regeneration treatment by using said regeneration flasher steam and/or said auxiliary heater, said high pressure and high temperature solid organic compound is sprayed into said low temperature and low pressure atomizing and dehumidifying granulation set to be atomized dehumidified and granulated, thus solid regenerated organic compound product is made.

9. The organic compound pyrolysis flashover energy-saving regeneration treatment system as claimed in claim 1, said remote monitoring and automatic adjustment unit includes a plurality of sensors provided respectively at said units, said sensors include but are not limited to: pressure sensors, flow sensors, liquid level sensors, temperature sensors and humidity sensors.
